(19) United States
(12) Patent Application Publication
(10) Pub. No.: US 2010/0310652 A1
(43) Pub. Date: Dec. 9, 2010

(54) COATED EXTENDED RELEASE PHARMACEUTICAL COMPOSITIONS OF LEVETIRACETAM

(75) Inventors: Rajesh Kshirsagar, Gujarat (IN); Ashwin Rao, Gujarant (IN); Atul Kathiriya, Gujarat (IN)

Correspondence Address: MERCHANT & GOULD PC, P.O. BOX 2903, MINNEAPOLIS, MN, 55402-0903, US

(73) Assignee: Alembic Limited, Vadodara ,Gujarat (IN)

(21) Appl. No.: 12/809,164

(22) PCT Filed: Dec. 22, 2008
(86) PCT No.: PCT/IN2008/000851

§ 371 Date: Jun. 18, 2010

(30) Foreign Application Priority Data
Dec. 21, 2007 (IN) 1849/MUM/2007

(51) Int. Cl. A61K 9/28; A61K 31/4015; A61K 9/36

(52) U.S. Cl. 424/465; 514/424; 424/472

(57) ABSTRACT

An extended release pharmaceutical composition comprising levetiracetam. A coated extended release pharmaceutical composition comprising levetiracetam wherein the core is coated with a rate controlling composition.

### FIELD OF THE INVENTION

The present invention relates to an extended release pharmaceutical composition comprising levetiracetam. In particular, the present invention relates to a novel coated extended release pharmaceutical composition comprising levetiracetam wherein the core is coated with a rate controlling composition.

### BACKGROUND OF THE INVENTION

Levetiracetam is chemically (−)-(S)-α-ethyl-2-oxo-1-pyrrolidine acetamide having molecular formula C₈H₁₄N₂O₂ and molecular weight of 170.21. It is a white to off white crystalline powder and has an aqueous solubility of 1.04 g/mL. Levetiracetam is indicated as adjunctive therapy in the treatment of partial onset seizures in adults with epilepsy. It is also indicated as adjunctive therapy in the treatment of myoclonic seizures in adults and adolescents 12 years of age and older with juvenile myoclonic epilepsy and as adjunctive therapy in the treatment of primary generalized tonic-clonic seizures in adults and children 6 years of age and older with idiopathic generalized epilepsy. It is marketed in the United States under the brand name Keppra® as 250 mg, 500 mg, 750 mg and 1000 mg tablets and as 100 mg/mL solution. Keppra® is also available for intravenous use as a 500 mg/5 ml injection for oral administration. Levetiracetam is a Class I molecule as per the Biopharmaceutics Classification System, since it is highly soluble (1.04 g/ml), highly permeable (F>90%) and more than 85% of the drug is released in 15 minutes in three different pH media.

Levetiracetam has a relatively low order of toxicity and a relatively high therapeutic index. The twice daily dosing regimen for immediate-release levetiracetam tablets is well tolerated but with few incidences of neuropsychiatric adverse events like somnolence, fatigue, coordination difficulties and behavioral abnormalities. These adverse events are proportionate to the drug plasma level and therefore there is a need in the art for an extended release once-daily regimen of levetiracetam.

WO 01/51033 provides for a Solid pharmaceutical compound that can be administered orally, permitting controlled release of at least one active substance which can be Levetiracetam consisting of a homogeneous mixture comprising active substance, at least one matrix excipient between 5 and 95% by weight in relation to total weight of the compound, selected among the inert matrices, the hydrophilic, or lipid matrices, mixtures of inert and lipidic matrices mixture of hydrophilic and inert matrices; at least one entero-soluble polymer between 2 and 50% by weight in relation to the total weight of the compound and at least one alkalinizing agent soluble in a aqueous phase under conditions of physiological pH, of at least 0.5 to 50% by weight in relation to the total weight of the compound.

WO 03/101428 provides for a method for the manufacture of a pharmaceutical compound with retarded release of the active principle, which can be Levetiracetam. A mixture of active substance and the polymer that provides the retarded release are compressed by putting them through two rollers that have a temperature of more than 40° C. and compaction force is exerted on it of more than 15 to 40 kN/cm roller width. The compressed mixture is powdered to the desired particle size and if required the process is repeated.

However, the high dose of levetiracetam requires a high amount of rate controlling excipients to be used in the formulation which increases the size of the dosage form thereby affecting patient comfort while swallowing it. Also levetiracetam is a freely water soluble drug and it is known in the art that, it is very difficult to develop a pharmaceutical composition with a sufficiently slow dissolution rate for freely soluble drugs.

The present invention provides a novel coated extended release pharmaceutical composition comprising levetiracetam which uses minimal amount of excipients in the core thereby minimizing the size of the dosage form.

The coating exhibits excellent elastic properties thereby avoiding dose dumping and also prevents the burst effect that is normally observed when formulating matrix extended release pharmaceutical compositions of highly soluble drugs like levetiracetam.

### OBJECT OF THE INVENTION

An object of the present invention is to provide a coated extended release pharmaceutical composition comprising levetiracetam for once daily dosing.

Another object of the present invention is to provide a process for preparation of a coated extended release pharmaceutical composition comprising levetiracetam.

### SUMMARY OF THE INVENTION

The present invention provides a coated extended release pharmaceutical composition comprising levetiracetam for once daily dosing

The present invention provides a process for preparation of a coated extended release pharmaceutical composition comprising levetiracetam.

### DETAILED DESCRIPTION OF THE INVENTION

The present invention provides for a novel coated extended release pharmaceutical composition comprising levetiracetam wherein the core is coated with a rate controlling composition. The term “extended release” for the purposes of this invention refers to release of an active pharmaceutical agent over a prolonged period of time, such as for example over a period of 8, 12, 16 or 24 hours. The term ‘extended release’ as herein used includes sustained release, modified release, delayed release and controlled release.

In a preferred embodiment, the pharmaceutical composition of the present invention comprises 50-99% of levetiracetam; preferably the present invention comprises 60-95% of levetiracetam or a pharmaceutically acceptable salt thereof

The pharmaceutical compositions of the present invention can be any solid dosage form for example, but not limited to, granules, pellets and tablets. The core dosage forms can be prepared by any of the means using excipients well known to the person skilled in the art.

In a preferred embodiment, the novel coated extended release pharmaceutical composition comprising levetiracetam is in the form of a tablet. The core of the coated extended release tablet composition comprises levetiracetam and minimum amount of conventional excipients.

The conventional excipients according to present invention are those excipients which are commonly used in the art and known to any person skilled in the art. These include, but are not limited to, fillers, binders, lubricants, plasticizers, glidants and the like.

Examples of fillers or diluents include, but are not limited to, corn starch, lactose, sucrose, microcrystalline cellulose, kaolin, mannitol, dextrose, lactose, sorbitol, dicalcium phosphate, calcium carbonate, sodium chloride, maltitol, xylitol and the like.

Examples of binders include, but are not limited to methylcellulose, hydroxypropylcellulose, hydroxyethyl cellulose, hydroxypropyl methylcellulose, sodium carboxymethylcellulose, polyvinylpyrrolidone, sucrose, starch, ethylcellulose, acacia, gelatin, gum arabic, copovidone, polyvinyl alcohol, pullulan, agar, tragacanth, sodium alginate, alginic acid, and the like. Glycerides such as for example mono-, di- or triglycerides: stearin, palmitin, laurin, myristin, hydrogenated castor or cottonseed oils, glyceryl palmitostearate, glyceryl behenate and the like, fatty acids and alcohols such as for example stearic, palmitic or lauric acids, stearyl, cetyl or cetosteryl alcohols and the like and waxes such as for example white wax, bees wax, carnauba wax and the like

Examples of lubricants and glidants include, but are not limited, to stearates and stearic acid, silicone fluid, talc, waxes, oils, colloidal silicon dioxide, sodium stearyl fumarate, polyethylene glycols, hydrogenated vegetable oil, glyceryl behenate, magnesium trisilicate, microcrystalline wax, yellow beeswax, white beeswax and the like.

It should be appreciated that there is considerable overlap between the above-listed additives in common usage, since a given additive is often classified differently by different practitioners in the field, or is commonly used for any of several different functions. Thus, the above-listed additives should be taken as merely exemplary, and not limiting, of the types of additives that can be included in compositions of the present invention. One or more of these additives can be selected and used by the skilled artisan having regard to the particular desired properties of the dosage form by routine experimentation without any undue burden. The amount of each type of additive employed may vary within ranges conventional in the art.

In a preferred embodiment, the core of the present invention is formulated with levetiracetam, a binder and a lubricant. In a more preferred embodiment, the core of the present invention is formulated with levetiracetam, polyvinyl pyrrolidone as the binder and magnesium stearate as the lubricant.

The core tablets comprising levetiracetam can be prepared by processes well known to those of skill in the art. For example, core tablets can be prepared by wet granulation, dry granulation, melt granulation and the like. In a preferred embodiment, the core tablets comprising levetiracetam are prepared by wet granulation.

In a further embodiment, the core tablets are prepared by melt granulation.

The core dosage forms comprising levetiracetam are then coated with a suitable rate controlling composition to control the release rate of levetiracetam. The rate controlling composition can comprise one or more hydrophilic agents and one or more hydrophobic agents.

Suitable hydrophilic agents include, but are not limited to water soluble polymers such as hydroxyethyl cellulose, hydroxypropyl cellulose, carboxymethyl cellulose, hydroxypropylmethyl cellulose, sodium carboxymethyl cellulose, vinylpyrrolidone/vinyl acetate copolymer for example marketed as Plasdone® S-630, polyvinyl alcohol, polyethylene glycol and the like. Saccharides such as monosaccharides, disaccharides, oligosaccharides, polysaccharides or sugar alcohols which include but are not limited to sucrose, xylitol, mannitol, sorbitol, glucose, fructose, galactose, maltitol, lactose, maltodextrin. Water soluble organic acids, water soluble salts of organic acids, water soluble organic bases, water soluble salts of organic bases which include but are not limited to citric acid or salts thereof, amino acids or salt thereof, inorganic salts such as sodium carbonate, sodium bicarbonate, potassium chloride and sodium chloride and the like

Suitable hydrophobic agents include, but are not limited to cellulose acetate, ethylcellulose, ammoniomethacrylate copolymers for example marketed under the brand name of Eudragit® RL, aminoalkyl methacrylate copolymers, for example, marketed under the brand name of Eudragit® RS, polyvinyl acetate for example marketed under the brand name Kollicoat® SR and the like.

In a preferred embodiment, the coating comprises of a combination of a hydrophilic agent and a hydrophobic agent. The ratio of the hydrophilic agent to the hydrophobic agent is between 1:5 to 5:1.

In a still preferred embodiment of the present invention, the coating comprises from about 2 to15% w/w of the core, more preferably the coating comprises from about 2 to 8% w/w of the core.

The coating composition may optionally contain other excipients which include, but are not limited to plasticizers, opacifiers, coloring agents and antifoaming agents. Examples of plasticizers include, but are not limited to citrates such as triethyl citrate, acetyl tributyl citrate, phthalates, dibutyl sebacate, triacetin, polyethylene glycol and the like.

Examples of opacifying agents and coloring agents include, but are not limited to titanium dioxide, talc, aluminum lake dyes, insoluble pigments, water-soluble dyes and the like. Antifoaming agents include, but are not limited to silicone, simethicone and the like.

The core tablets can be coated using any of the techniques well known to the persons skilled in the art. In a preferred embodiment, coating of core tablets of levetiracetam is carried out by spraying a non-aqueous dispersion of the coating composition excipients onto a core tablet bed in a perforated coating pan.

The extended release properties of the pharmaceutical composition of the present invention may be demonstrated by monitoring the dissolution of the active ingredient. The dissolution of the active ingredient may be monitored using standard procedures well known to those skilled in the art (e.g. the dissolution test procedures, such as the Rotating Basket Method (Apparatus I) or Paddle Method (Apparatus II), disclosed in the U.S. Pharmacopeia (USP). Such procedures include those in which the formulation is immersed in an aqueous medium such as water or hydrochloric acid and aliquots of the medium are withdrawn at various time points over a period of 24 hours. The aliquots are analyzed using high pressure liquid chromatography (HPLC) with UV detection to determine the concentration of dissolved active ingredient using standard methodology.

In a particular embodiment, the dissolution profile is determined by the Rotating Basket method by immersing a tablet in 900 ml of pH 6.8 buffer at a speed of 100 rpm.

As mentioned above, levetiracetam exhibits useful antiepileptic activity and therefore the formulations of this invention may be used, for example, in the treatment for seizures.

While the present invention has been described in terms of its specific embodiments, certain modifications and equivalents will be apparent to those skilled in the art and are intended to be included within the scope of the present invention. The invention may be further illustrated by the following non-limiting examples:

### Example 1

| Ingredients | Weight (mg) |
|---|---|
| Levetiracetam | 500.0 |
| Polyvinyl pyrrolidone K 30 | 10.0 |
| Purified water | q.s |
| Magnesium stearate | 5.1 |
| Total | 515.1 |

Procedure:

Levetiracetam is granulated with aqueous solution of polyvinyl pyrrolidone and dried. The granules obtained are sifted, lubricated with magnesium stearate and compressed into tablets using 16.5×8 mm capsule shaped punches to give a tablet of 515.1 mg.

| Coating | % w/w |
|---|---|
| Ethyl cellulose (Ethocel, 7cps) | 65 |
| Polyethylene glycol | 35 |
| Total | 100 |

Ethyl cellulose is dispersed in isopropyl alcohol and kept for stirring for one hour. Dichloro methane is then added to this dispersion. Polyethylene glycol is dissolved in water and added to the ethyl cellulose dispersion to make the coating solution which is then sprayed onto the tablets upto a weight build up of the dry coating up to 6% w/w of the tablet weight.

### Example 2

| Ingredients | Weight (mg) |
|---|---|
| Levetirecetam | 500 |
| Glyceryl Behenate (Compritol 888 ATO) | 150 |
| Magnesium Stearate | 08 |
| Total | 658 |

Procedure:

Levetirecetam and Glyceryl behenate were mixed properly and melt granulated. After cooling, the mass was sifted and lubricated with magnesium stearate. The lubricated blend was subjected for compression to get uncoated tablets. The core is then coated with the following coating solution.

| Coating | % w/w |
|---|---|
| Ethyl cellulose (Ethocel, 7cps) | 52 |
| Polyethylene glycol | 20 |
| Hypromellose (Methocel E3 LV) | 28 |
| Total | 100 |

Ethyl cellulose and hypromellose (Methocel E3 LV) is dispersed in isopropyl alcohol and kept for stirring for one hour. Dichloromethane is then added to this dispersion. Polyethylene glycol is dissolved in water and added to the ethyl cellulose dispersion to make the coating solution which is then sprayed onto the tablets up to a weight build up of the dry coating up to 6% w/w of the tablet weight.

Dissolution Studies Data:

Apparatus: USP Type I—Basket, 100 rpm

| | % release of API in | % release of API in |
|---|---|---|
| Time (hrs) | Example 1 | Example 2 |
| 1 | 9 | 18 |
| 2 | 19 | 36 |
| 4 | 39 | 49 |
| 6 | 55 | 74 |
| 8 | 69 | 84 |
| 10 | 81 | 91 |
| 12 | 88 | 96 |

The results above shows that the percentage release in the initial 2 hours is only 19% in example 1 and 36% in example2. This indicates that there is no dose dumping.

1. A coated extended release pharmaceutical composition comprising levetiracetam wherein the core is coated with a rate controlling composition.

2. A coated extended release pharmaceutical composition according to claim 1, wherein rate controlling composition comprises one or more hydrophilic agents and one or more hydrophobic agents.

3. A coated extended release pharmaceutical composition according to claim 1, wherein hydrophilic agent can be a water soluble polymer, saccharides such as monosaccharides, disaccharides, oligosaccharides, polysaccharides or sugar alcohols, water soluble organic acids or salts thereof, water soluble organic bases or salts thereof and inorganic salts.

4. A coated extended release pharmaceutical composition according to claim 3, wherein hydrophilic agent is selected from hydroxyethyl cellulose, hydroxypropyl cellulose, carboxymethyl cellulose, hydroxypropylmethyl cellulose, sodium carboxymethyl cellulose, vinylpyrrolidone/vinyl acetate copolymer for example marketed as Plasdone® S-630, polyvinyl alcohol, polyethylene, sucrose, xylitol, mannitol, sorbitol, glucose, fructose, galactose, maltitol, lactose, maltodextrin, citric acid or salts thereof, amino acids or salts thereof, sodium carbonate, sodium bicarbonate, potassium chloride and sodium chloride.

5. A coated extended release pharmaceutical composition according to claim 1, wherein hydrophobic agent is selected from cellulose acetate, ethylcellulose, ammoniomethacrylate copolymers, aminoalkyl methacrylate copolymers, polyvinyl acetate.

6. A coated extended release pharmaceutical composition according to claim 1, wherein the composition comprises 50-99% w/w of levetiracetam.

7. A coated extended release pharmaceutical composition according to claim 1, wherein the composition comprises 60-95% w/w of levetiracetam.

8. A coated extended release pharmaceutical composition according to claim 1, wherein the tablet is coated from about 2 to 15% w/w of the core.

9. A coated extended release pharmaceutical composition according to claim 1, wherein the tablet is coated from about 2 to 8% w/w of the core.

10. A coated extended release pharmaceutical composition of levetiracetam according to claim 1, which is administered once daily.

11. A process for preparation of a coated extended release pharmaceutical composition according to claim 1, comprising
i) mixing levetiracetam and optionally one or more excipients
ii) granulating the mixture, and sifting and lubricating the obtained granules
iii) compressing the sifted and lubricated granules into tablets and
iv) coating above tablets with a rate controlling composition.

12. (canceled)
